(19) United States
(12) Patent Application Publication
(10) Pub. No.: US 2026/0279542 A1
(43) Pub. Date: Sep. 17, 2026

(54) RETRAINING DORSAL VISUAL PATHWAYS IMPROVES COGNITIVE SKILLS and IMPROVES EXECUTIVE CONTROL NETWORKS

(71) Applicant: Teri Lawton, Solana Beach, CA (US)

(72) Inventor: Teri Lawton, Solana Beach, CA (US)

(21) Appl. No.: 19/390,526

(22) Filed: Nov. 15, 2025

Related U.S. Application Data
(60) Provisional application No. 63/771,630, filed on Mar. 14, 2025.

(51) Int. Cl. G16H 20/70; G16H 40/67

(52) U.S. Cl. CPC G16H 20/70; G16H 40/67

(57) ABSTRACT

In one embodiment, a method to improve cognitive skills of a user includes displaying a background and a test pattern. The background includes a pattern with visibly lighter and darker portions that vary in luminance across a spatial dimension at multiple spatial frequencies. The test pattern includes a contrast between lighter and darker portions moving within the background boundaries. The method receives a communication from the user through a user interface indicating the directions the test pattern appears to the user and receiving feedback if incorrect. The method repeats the displaying and receiving steps a predetermined number of pattern sequences. The method determines when the predetermined number of pattern sequences is complete and presents a predetermined sequence of digits. The method obtains a communication wherein the user enters the predetermined sequence of digits in order and corrects the entries based at least in part on the correct order.

### CROSS REFERENCE TO RELATED PATENT APPLICATIONS

This patent application is a continuation of U.S. Provisional Patent Application No. 63/771,630 filed on Mar. 14, 2025 and is related to U.S. Pat. No. 8,979,263 filed on Apr. 5, 2010 and U.S. Pat. No. 6,213,956 filed on Jan. 24, 2000. Both patents are incorporated herein by reference in their entirety for all purposes.

### BACKGROUND

Traumatic brain injury (TBI) is an injury to the brain typically caused by a violent jolt or blow to the head or body of a person. While some brain injuries may be mild with few lasting symptoms, a TBI may include cognitive, behavioral, or mental symptoms. These symptoms may include loss of consciousness, a state of daze or confusion, memory or concentration problems, mood changes, mood swings, depressive feelings or anxiousness, difficulty sleeping, and increased sleeping.

Mild TBIs (mTBI) are prevalent, with over 2.5 million concussions occurring in the U.S. each year and only 1 in 6 of these concussions being diagnosed. Concussions represent 80% of TBIs, with the bulk of concussions resulting from motor vehicle accidents, falls, and situations involving sudden acceleration and/or deceleration of the head, such as sports injuries. Individuals suffering from a mTBI currently have limited treatment options. Currently, there are no proven solutions to remediate cognitive deficits prevalent in those with a mTBI. The current cognitive deficits treatment programs are neither robust nor consistent. The programs do not improve the executive functions and attention in mTBI.

### BRIEF SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one embodiment, a method to improve cognitive skills of a user is disclosed. The method includes displaying, on a monitor, a background and a test pattern. The background includes a pattern which has visibly lighter portions and darker portions that visibly vary in luminance across a spatial dimension at multiple spatial frequencies. The test pattern includes a contrast between a lighter portion and a darker portion moving in one or more directions within the boundaries of the background. The method includes receiving a communication from the user through a user interface indicating the one or more directions in which the test pattern appears to the user to have moved. The method also includes providing feedback to the user when an incorrect communication is received and repeating the displaying and the receiving steps associated with the background and the test pattern a predetermined number of pattern sequences. The method further includes determining when the predetermined number of pattern sequences is complete and presenting, on the monitor, a predetermined sequence of digits to the user when the predetermined number of pattern sequences is complete. The method further includes obtaining a communication from the user through the user interface wherein the user enters the predetermined sequence of digits in the order presented and correcting the entries based at least in part on the correct order of sequence of digits displayed.

In some embodiments, each digit may be separated by at least 10 msec. In some embodiments, the method may also include presenting, on the monitor, a further set of predetermined sequence of digits to the user after a prior set of predetermined sequences of digits is complete, where the further set of predetermined number of digits may increase by one digit each time, the further set of predetermined sequences of digits may end when a total of 10 digits is presented. In some embodiments, each further set of predetermined sequence of digits may present no repeating digits and no repeating sequences. In some embodiments, a quantity of the further set of predetermined sequence of digits may one greater than a quantity of the prior set of predetermined sequence of digits.

In still further embodiments, the method may include presenting a second predetermined sequence of digits to the user after the first entries are corrected. In some embodiments, the test pattern may be displayed in low contrast grayscale below 10% contrast, and the background pattern contrast may be one of equal to 20% contrast and below 20% contrast. In some embodiments, the method may include receiving a request from the user to repeat a process, wherein the process may be one of the pattern sequence and digit sequence. In some embodiments, one of the pattern sequence and digit sequence process may repeat at a different complexity level.

In some embodiments, the method may record user entries in the associated user profile on the encrypted server, wherein the entries may be stored in a structured hierarchical format. In some embodiments, the method may compile the user personal information and user entries into a dashboard. In further embodiments, the method may enable a second user to remotely access the pattern and digit sequence presenting and user response.

In some embodiments, the method may calibrate the monitor to test the contrast of the lighter portion and the darker portion of the test pattern. The method may also display the contrast pattern in 12-bit color grayscale on the monitor. The method may also include converting 8-bit color grayscale monitor to display the contrast pattern in 12-bit color grayscale.

In another embodiment, a method to remediate cognitive skills is disclosed. The method includes presenting, on a monitor, a predetermined sequence of digits to a user, obtaining a communication from the user through a user interface wherein the user enters the predetermined sequence of digits in the order presented, and correcting the entries based at least in part on the correct order of sequence of digits displayed.

In some embodiments, the predetermined number of digits may be five digits, each digit may be separated by at least 10 msec. The digits may increase by one digit each time up to a total of 10 digits, may present no repeating digits and no repeating sequences. In some embodiments, the method may include determining when the user has completed a predetermined number of sequence recounting cycles and ending the training when the predetermined number of sequence recounting cycles is completed. The method may also include displaying a second set of predetermined sequence of numbers to the user when the user has not completed the predetermined number of sequence recounting cycles.

In another embodiment, a method to improve cognitive skills of a user is disclosed. The method includes displaying, on a monitor, a background and a test pattern, wherein the background includes a pattern which has visibly lighter portions and darker portions that visibly vary in luminance across a spatial dimension at multiple spatial frequencies, and wherein the test pattern includes a 12 bit grayscale contrast between a lighter portion and a darker portion moving in one or more directions within the boundaries of the background. The method includes receiving a communication from the user through a user interface indicating the one or more directions in which the test pattern appears to the user to have moved. The method includes providing feedback to the user when an incorrect communication is received and repeating the displaying and the receiving steps associated with the background and the test pattern a predetermined number of pattern sequences. The method also includes determining when the predetermined number of pattern sequences is complete and presenting, on the monitor, a predetermined sequence of digits to the user one digit at a time, displaying each digit for 500 ms, wherein the predetermined sequence of digits is displayed in black lettering on a white background, when the predetermined number of pattern sequences is complete. The method further includes obtaining a communication from the user through the user interface wherein the user enters the predetermined sequence of digits in the order presented and correcting the entries based at least in part on the correct order of sequence of digits displayed.

### BRIEF DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a diagram of a sample system in accordance with the present disclosure;

FIG. 2 is a block diagram of an example neurotraining module in accordance with one example of the present disclosure;

FIG. 3 is an exemplary illustration of patterns in accordance with one example of present disclosure;

FIG. 4 is an exemplary flow diagram in accordance with embodiments of the present disclosure;

FIG. 5 is an exemplary flow diagram in accordance with embodiments of the present disclosure;

FIG. 6 is an exemplary chart of improvements in cognitive skills in accordance with embodiments of the present disclosure;

FIG. 7 is an exemplary illustration of pre and post MEG working memory, executive control, attention, and dorsal visual processing networks in accordance with embodiments of the present disclosure;

FIG. 8 is a is a block diagram of an example device in accordance with one example of the present disclosure.

FIG. 9 is an exemplary graph of contrast sensitivity scores in accordance with embodiments of the present disclosure;

FIG. 10 is an exemplary table of direction discrimination contrast thresholds in accordance with embodiments of the present disclosure

FIG. 11 is another exemplary graph of direction discrimination threshold in accordance with embodiments of the present disclosure;

FIG. 12 is an exemplary chart of reading speeds in accordance with embodiments of the present disclosure;

FIG. 13 is an exemplary graph of reading speeds in accordance with embodiments of the present disclosure;

FIG. 14 is an exemplary chart of stories that may be read to determine reading speed with embodiments of the present disclosure;

FIG. 15 is an exemplary chart of rewards to user in accordance with embodiments of the present disclosure; and

FIG. 16 is an exemplary chart of user dashboard in accordance with embodiments of the present disclosure.

### DETAILED DESCRIPTION

The detailed description set forth below in connection with the appended drawings, where like numerals reference like elements, are intended as a description of various embodiments of the present disclosure and are not intended to represent the only embodiments. Each embodiment described in this disclosure is provided merely as an example or illustration and should not be construed as precluding other embodiments. The illustrative examples provided herein are not intended to be exhaustive or to limit the disclosure to the precise forms disclosed.

In the following description, specific details are set forth to provide a thorough understanding of exemplary embodiments of the present disclosure. It will be apparent to one skilled in the art, however, that the embodiments disclosed herein may be practiced without embodying all of the specific details. In some instances, well-known process steps have not been described in detail in order not to unnecessarily obscure various aspects of the present disclosure. Further, it will be appreciated that embodiments of the present disclosure may employ any combination of features described herein.

Substantial research reveals that the effects of a mild traumatic brain injury (mTBI) reflect disruptions of the neural networks for cognitive control. Working memory (WM), attention, and other executive function deficits are prominent cognitive sequelae of mTBI. WM deficits in mTBI do not improve over time. Thus, daily living may be dramatically impaired after a mTBI. Visual timing deficits, resulting from magnocellular (motion-processing) deficits can be persistent in individuals with a mTBI, which may manifest as timing deficits in the dorsal pathways, and attention and executive control networks. Many aspects of cognitive control deficits in those with a mTBI may in fact result from neural timing deficits. Compensation for timing issues by increased prefrontal cortical recruitment may manifest as increased distractibility, WM deficits, and problems with balance and coordination. This expended effort may underlie fatigue, headache, irritability, anxiety, and depression.

The disclosure relates to the remediation of a fundamental visual timing deficit affecting motion discrimination at a low level of cognitive processing that generalizes to high level cognitive skills (attention and working memory) reliant upon motion processing as a foundation.

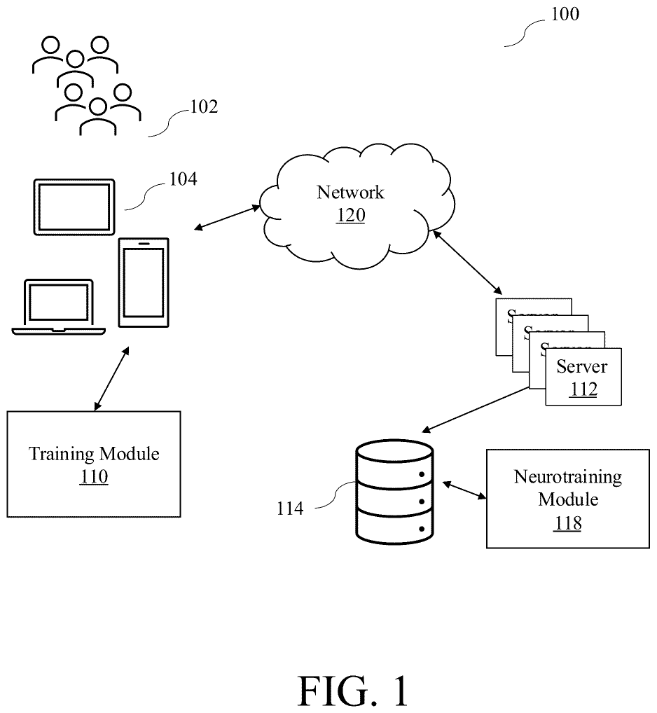

FIG. 1 is a block diagram illustrating one embodiment of an environment 100 in which the present systems and methods may be implemented. The environment 100 may include one or more users, consumers, patients, and the like 102, one or more devices 104 associated with the users, one or more databases or servers 112, and a network 120 that allows the different parts of the system 100 to communication with one another. In some embodiments, the user 102 may be a patient experiencing mTBI symptoms. In another embodiment, the user 102 may be a person looking to improve their brain function.

Examples of the device 104 may include a mobile phone, a laptop, a desktop computer, a tablet, mobile computing device, smart phone, personal computing device, computer, server, etc. The device may allow a user to connect with various components of the system and utilize the neurotraining module 118 or training module 110. The device 104 has a visual and audio output system for interactive feedback as well as an input system.

Examples of the server 112 may include a server administered by a medical device company or another company that may provide neurotraining to patients or users. In some embodiments, the system 100 may include a single server or multiple servers 112. The server(s) 112 may share data and share resources and distribute work. The servers 112 and devices and communication streams all comply with HIPAA protocols and procedures.

In some embodiments, devices 104 may communicate with servers 112 via network 120. Examples of a network 120 include cloud networks, local area networks (LAN), wide area networks (WAN), virtual private networks (VPN), wireless network, cellular networks (using 3G, 4G, and/or LTE, for example), etc. In some configurations, the network 120 may include the internet. In some embodiments, devices 104 may include an application that interfaces with one or more functions of neurotraining module 118 or the training module 110 or both. The training module 110 may be a local program application operable without an internet connection. In some embodiments, the user may input confidential and sensitive information without worry that the information may be on a public server that others besides those authorized by user may access. For example, since all data is HIPAA encrypted, it is only accessible by therapists or administrators. In the current embodiments, the user 102 may use the device 112 to connect to the neurotraining module 118 via a webpage, application, or the like. In these embodiments, the neurotraining module 118 has dedicated confidential and protected databases specific to clients.

For example, in some embodiments, server 112 may be coupled to database 114. Database 114 may optionally include a neurotraining module 118. In still further embodiments, the device 104 may access the neurotraining module 118 via the server 112. Database 114 may be internal or external to the server 112. In one example, a device 104 may be coupled directly to database 114, database 114 being internal or external to device 104. The neurotraining module 118 may comprise the software and data necessary to implement the disclosure disclosed herein including at least tracking patient data and administering training.

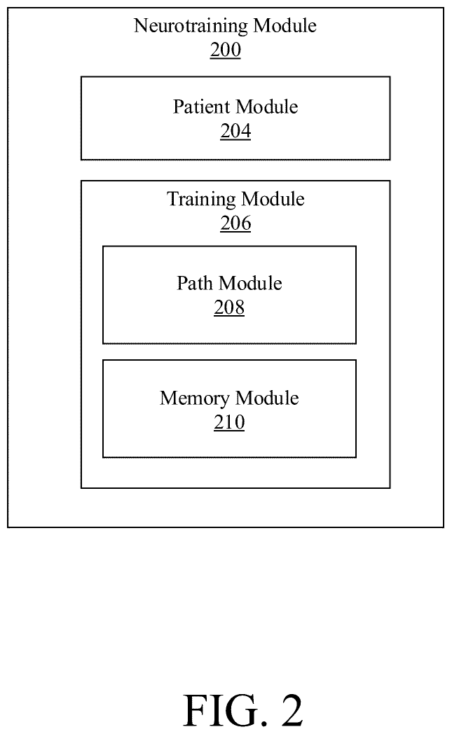

FIG. 2 is a block diagram illustrating exemplary components of one example of a neurotraining module 200. The neurotraining module 200 may include a patient module 204, and a training module 206. The training module 206 may include a PATH module 208 and a memory module 210.

The patient module 204 may store all patient information including health information, previous trauma, initial screening test scores, and the like. The patient module 204 may incorporate information gathered at patient intake such as demographics. In some embodiments, the patient portal may collect information on the client's weaknesses or deficits. This may enable the patient module 204 to initiate a pre and post training intake to gauge the patient's improvement in their area for growth. The patient module 204 may comply with all HIPPAA requirements.

In some embodiments, the patient module 204 may also track training and successes, learning moments, failures, and the like of the patient. This information may include gaming features such as collecting tokens for completing levels, collecting the same or different tokens for increased difficulty, efficiency, and the like.

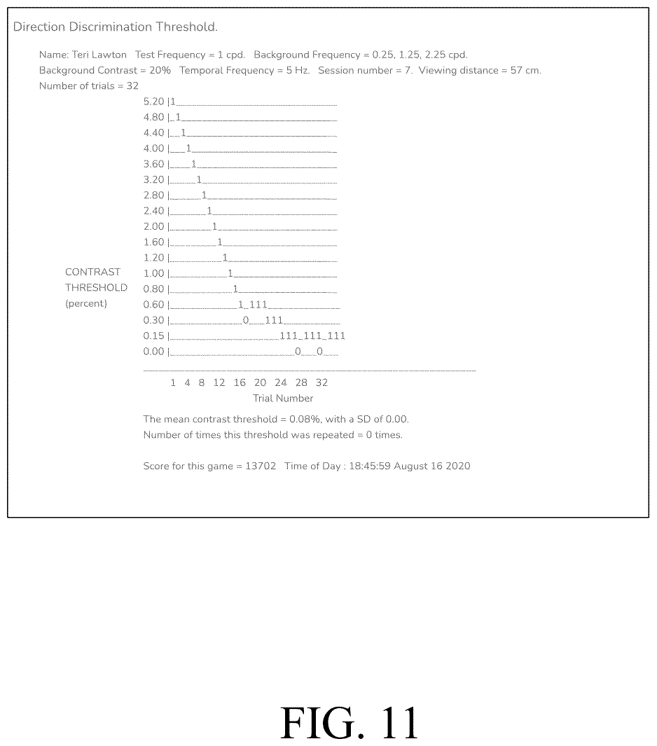

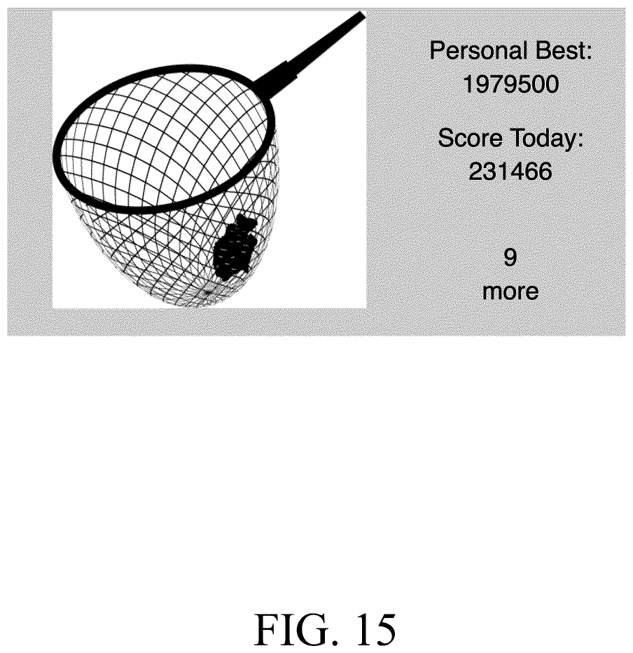

In some embodiments, the patient module 204 may also track and, in some instances, display graphics to the user to display progress throughout the training program. For example, as shown in FIG. 11, the patient module 204 may track an initial testing in a specific functioning area and display the patient's progress in that area. This may enable the patient to review their progress and feel rewarded for their efforts. While the graph in FIG. 11 shows the data for contrast sensitivity function, the patient module 204 may track and graph multiple different data points including progress discriminating direction of movement for each pattern as complexity level increases, making task more challenging, and for both Motion and Motion Memory programs, and progress on both reading speed and digit memory tasks. Similarly, the patient module 204 may reward the progress throughout training by providing rewards for each level/pattern completed. The reward may be a token or other collectible for the user to track their progress throughout the training, see e.g. FIG. 15 discussed herein.

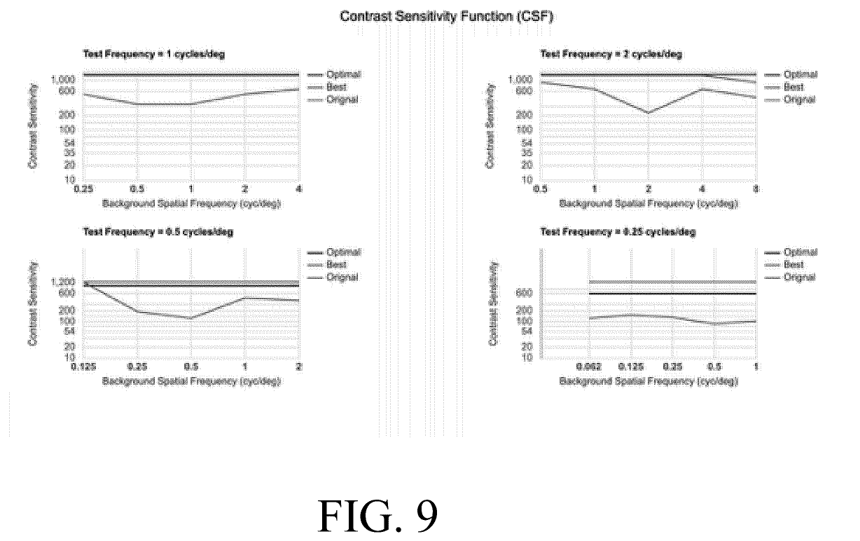

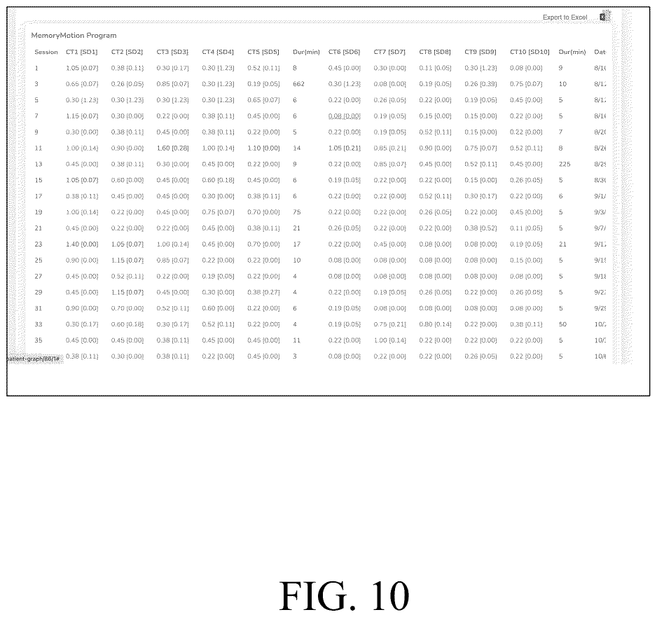

In some embodiments, as shown in FIG. 9, the patient module may track the patient's contrast value thresholds. In further embodiments, as shown in FIG. 11, the patient module 204 may track the patient's responses to testing and provide feedback data. FIG. 10 shows the contrast thresholds for each pattern as complexity level increases, increasing task difficulty. In some embodiments, the contrast thresholds may be color coded to differentiate which contrast thresholds are too high for maximum effectiveness (coded in red), are in the middle (coded in blue), being above approximately 1% contrast, helpful but not as beneficial as low contrast thresholds (coded in green) that are below approximately 1% contrast. In the example shown, each column displays a contrast threshold for each pattern as complexity level increases. This may be displayed in different arrangements. The data provided may allow the user to view potential improvements in increasing dorsal stream function, by measuring low contrast

### Thresholds.

In some embodiments, when a contrast threshold value is selected, a graph used to measure this contrast threshold may be displayed. One example of this graph is shown in FIG. 11. In the example shown, FIG. 11 may display a contrast threshold on the y column and a number of trials on the x column. The contrast threshold may be the minimum contrast needed to see direction of movement approximately 79% of the time. Overall, in some embodiments, FIG. 11 may display a reliability the contrast threshold that is not only showing the contrast threshold coupled with a number of trials to reach this contrast threshold. In some embodiments, a lower number of trials may be beneficial in terms of the time to complete task, and reliability of threshold measurement. In some embodiments, a low Standard Deviation (SD) may show that this is a more reliable threshold measurement. The low contrast threshold may imply that task improves brain function more than a high contrast threshold. In some instances, the graph may also include a date and time of the recording, provide more information about reliability of contrast threshold measurement, in addition to the pattern description, number of trials, and number of times this threshold had to be repeated, before this measurement.

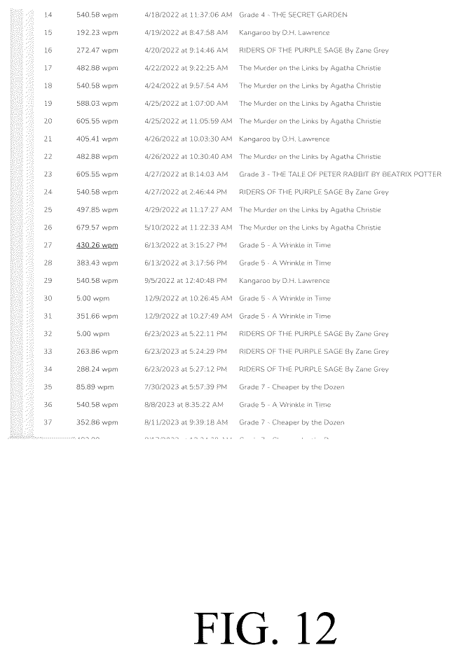

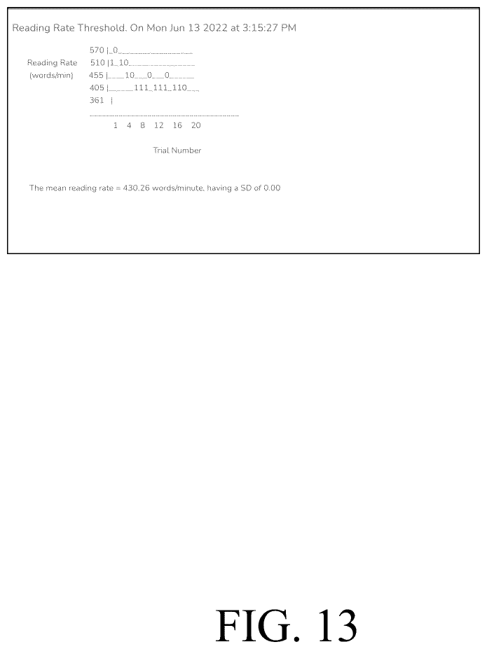

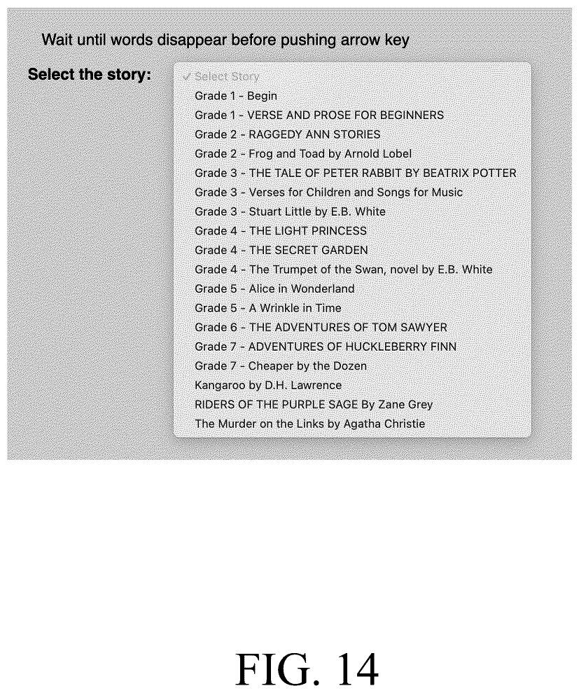

In some embodiments, the patient module 204 may also track and record reading rates. For example, as shown in FIGS. 12 and 13, the patient module 204 may track reading rates as words per minute for specific books, sections of books, paragraphs, readings, and the like. See FIG. 14 for an example of different books that may be read. Referring back to FIG. 2, in some instances, the patient module 204 may also track the date and time of the recording and, in some embodiments, link a reading rate or a digit memory performance rate to a specific point in training for the patient. The patient module 204 may then present subsequent text from the same reading story, or subsequent sequences of 5-10 digits, for example, to the patient after a predetermined period of training has been completed to track the patient's improvement in reading or remembering over time through the use of the training modules.

The training module 206 includes the PATH module 208 and the digit memory module 210. One example of the PATH module 208 is described in more detailed with respect to U.S. Pat. No. 8,979,263. In some embodiments, the PATH module 208 may be designed to activate the dorsal stream low levels (visual motion areas), improving visual timing and movement discrimination, and at high levels, improving attention, cognitive flexibility, processing speed, and working memory, improving high-level cognitive skills. Therefore, in some instances, the PATH module 208 may improve cognitive abilities not related to movement discrimination and may improve everyday cognitive performance and quality of life in mTBI, children, adults, and older adults. These improvements in visual timing may enable the attention and executive control networks to function better. PATH training in mTBI patients is the first time that improving low-level visual timing deficits in the dorsal stream were found to improve high-level cognitive functioning, both behaviorally and using a biomarker, MagnetoEncephaloGraphy (MEG) physiological brain recordings. Studies have shown PATH training had a much larger effect size for improving visual working memory of d=1.16 than found previously in a meta-analysis, d=0.2, which included data from 115 studies and 2,104 participants. Moreover, PATH training may be more rapid, taking only 5-10 hours, and more effective in improving cognitive skills, than competitive methods. PATH training also increases in complexity, progressing from slower to faster movements, and from one direction to several directions of movement, being an adaptive technology, which may be essential for effective brain training. Furthermore, evidence shows improvements in cognitive skills after PATH training are sustained over time.

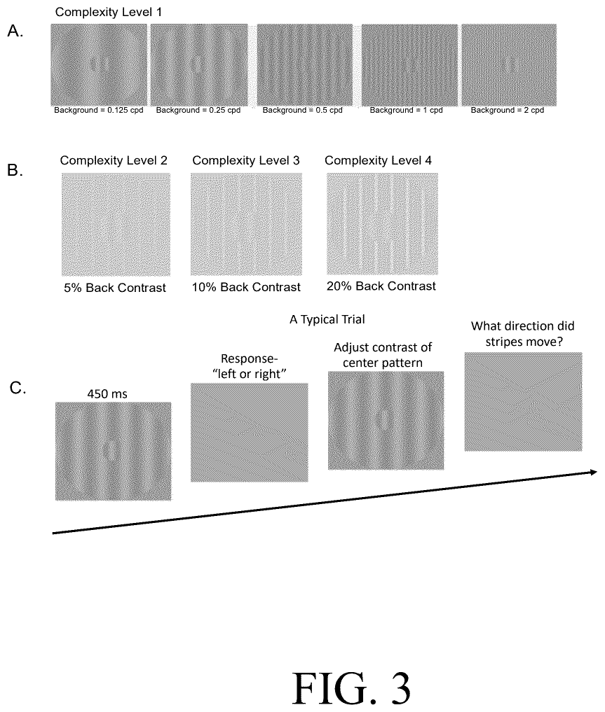

Referring to FIG. 3, FIG. 3 illustrates sample patterns for training at select complexity levels that the PATH module 208 may utilize. FIG. 3A illustrates a sample pattern for a beginning level of complexity. FIG. 3B illustrates sample patterns for increasing complexity levels above a beginning level. FIG. 3C illustrates the sequence of pattern flashes on a screen during a single trial.

For example, FIG. 3 displays visual timing intervention, implemented using the training module 206. This may remediate dorsal stream function, consisting of motion direction-discrimination training. In some embodiments, the mechanism that causes neural timing improvements may be the repeated viewing of stimuli in FIG. 3 moving left or right at increasing speeds relative to a stationary background, with sensitivity increasing the more PATH module 208 exercises are done.

In some embodiments, during the training presented by the PATH module 208, the bars in the “fish-shaped” window in the center of the screen formed by a sinusoidal grating, moved left or right very briefly. This brief period may consist of a time period of approximately 200-600 milliseconds. In further embodiments, this may comprise approximately less than or equal to 450 to 500 ms. When the pattern ends and the screen goes blank, the user may have the option to signal the direction the center pattern moved by pushing the left or right arrow key or another input directive (FIG. 3C). In some embodiments, the PATH module 208 may present a brief tone after incorrect responses, or another indicator that an answer was incorrect. In still further embodiments, the PATH module 208 may increase the contrast of center pattern at least one step. As such, in some embodiments, the PATH module 208 may adaptively change the contrast of the test pattern to keep the user at approximately 75 to 85% accurate. In further embodiments, the PATH module 208 may aim for approximately 79% correct. In some embodiments, the PATH module 208 may perform a full PATH training cycle (one complexity level) on each visit, for approximately fifteen to twenty-five minutes (one training cycle) followed by the Working Memory exercises described with reference to the memory module 210 for approximately five to fifteen minutes for a total of approximately twenty to forty minutes. In further embodiments, the PATH training cycle may last approximately twenty minutes, and the memory training may last approximately ten minutes for a total of approximately thirty minutes.

FIG. 3A displays sample patterns for PATH module 208 training at a low complexity level. In some embodiments, the low complexity level, called complexity level one for simplicity, may comprise a series of background frequencies spanning several octaves and test frequencies. The background frequencies may determine the background contrast present in the pattern displayed. The spatial frequency may comprise a measure of how often the pattern repeats. In some embodiments, the complexity level one may include a series of changes in increasing difficulty across the range of background spatial frequencies at different speeds, i.e. different temporal frequencies. For example, one routine for a level one complexity may include a background fundamental frequency two octaves lower in spatial frequency than the test frequency, one octave lower in spatial frequency than the test frequency, equal in spatial frequency to the test frequency, one octave higher in spatial frequency than the test frequency, and two octaves higher in spatial frequency than the approximately 0.5 cycle/degree “fish shaped” test pattern. In some embodiments, this series of testing may be presented in this order for each of the 4 test spatial frequencies. In other embodiments, the test spatial frequencies may vary. In some embodiments, the series of testing may have increasing pattern frequencies increasing from approximately 0.25 cycle/degree up to 2 cycles/degrees. In still further embodiments, across four test series, the frequency may increase from approximately 0.25 cycles/degree to approximately 0.5 cycle/degree, to approximately 1 cycle/degree, and ending on approximately 2 cycle/degree.

FIG. 3B displays increasing complexity levels, called complexity levels two, three, and four for simplicity. These increasing complexity levels have multifrequency backgrounds for center pattern as shown in FIG. 3B. The example shown includes the same background combination of spatial frequencies at 5%, 10% and 20% background contrast. In some instances, the increasing background frequencies may include the same fundamental frequency as in complexity level one, with a difference frequency equal to the test frequency. The increasing complexity may include increasing the background contrast from approximately 5% to approximately 10% to approximately 20% contrast. These contrast increases are exemplary and may be determined by the properties of the magnocellular neurons which may be optimal at 5% contrast, saturate at 10% contrast, with 20% contrast being the highest contrast that can be used. This high level of contrast may require integrating output from both magnocellular and parvocellular neurons to discriminate the direction of movement.

FIG. 3C illustrates an exemplary trial for PATH module 208 training. In the example shown, a pattern flashes on screen for approximately less than or equal to 450 msec while center stripes move left or right. When the screen goes blank, the PATH module 208 may wait for a user to depress the left or right arrow key (or another indicator). If the user is incorrect, a short tone or other indicator may sound. After the user inputs the directional motion of the sinewave grating (dim gray stripes), the next pattern with same or different contrast may flash on the screen while center stripes move left or right. In some embodiments, this sequence of patterns may be presented continuously until the contrast threshold for this pattern is measured. At the end of each contrast threshold measurement, a fishnet may appear with a fish rewarding the user, as shown in FIG. 15. For example, the user may earn a reward for each pattern having a contrast threshold approximately less than or equal to 1% contrast, personal best score, current score, number of patterns remaining, or some combination thereof. In some instances, the next pattern combination may be presented to measure the next contrast threshold until the PATH module 208 has presented all neurotraining patterns. The PATH module 208 may then present a ‘Thank You’ to the user indicating the completion of training for that period. In some embodiments, the PATH module 208 may also present a star for each level of complexity completed, showing a graph with the contrast threshold function (optimal, current, initial) for each test frequency with its five background patterns, e.g. see FIG. 9, or some combination thereof.

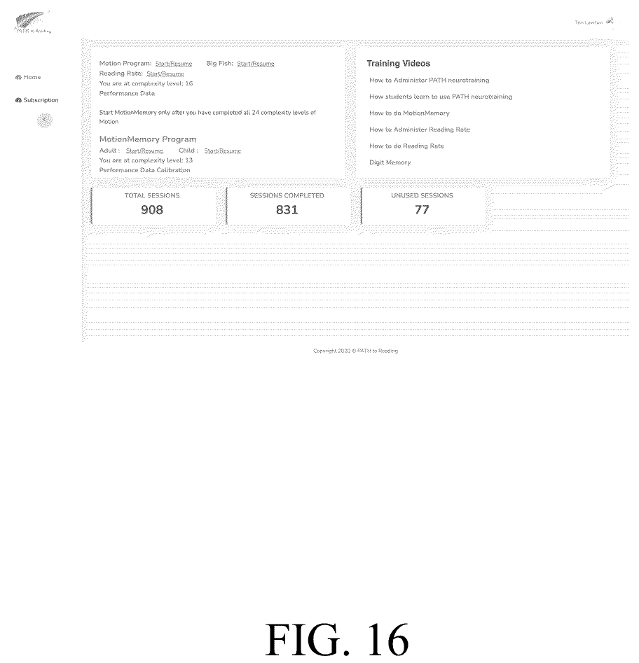

In some embodiments, at the start of a session, the PATH module 208 may set both the test and background gratings to approximately 5% contrast. In some embodiments, this may ensure the contrast of the test pattern is in the middle of the magnocellular contrast range. In some embodiments, the contrast and mean luminance, which may be approximately 120 cd/m2, may be measured using a Pritchard 1980A Spectra photometer. In some embodiments, the PATH module 208 may include a Calibrate tab, see FIG. 16, which may enable one to measure the desired pattern of varying contrast from approximately 0% up to approximately 30% contrast, one step at a time. In some embodiments, each time the user correctly identifies the direction the fish stripes move, the PATH module 208 may lower the contrast of the test grating one step until the user provides an incorrect answer.

In some embodiments, once the PATH module 208 receives an incorrect response, the PATH module 208 may present a double-staircase procedure. In some embodiments, the double staircase procedure may require the user to signal direction of movement correctly a sequential predetermined number of times before the contrast of test pattern is lowered. In some embodiments, the sequential predetermined number of times may be between two and five. In other embodiments, the sequential predetermined number of times may be approximately three. The sequential predetermined number may measure the movement-discrimination contrast threshold. Lowering a subject's contrast threshold may increase a subject's sensitivity to movement. In some embodiments, the staircase procedure may estimate the contrast threshold by using the most sensitive, repeatable measurements of contrast sensitivity possible. In some embodiments, a full training cycle of the movement-discrimination task required a predetermined number of contrast threshold determinations. In some embodiments, the full training cycle may use twenty contrast threshold determinations for each of the four test spatial frequencies (approximately 0.25, 0.5, 1, and 2 cyc/deg) paired with each of the five background spatial frequencies (equal to test frequency or approximately ±1 or ±2 octaves from the test frequency), patterns chosen to optimally active magno-cells.

In some embodiments, the PATH module 208 may introduce levels of difficulty by changing the background pattern, see center pattern in FIG. 3A. The background and foreground may have different patterns and spatial frequencies. In some embodiments, the difficulty may increase by increasing the pattern's complexity level, by increasing the number of directions of movement from one to two directions of motion, or some combination thereof. In some embodiments, the complexity level may increase the number of sine wave components in the background from one (FIG. 3A) to three harmonically related frequencies having a difference frequency equal to the test frequency (FIG. 3B). This may facilitate movement discrimination by providing a wider background frame of reference. In further embodiments, the complexity level may increase by changing the background contrast from approximately 5% to approximately 10% to approximately 20%. This may increase the amount of parvocellular activity, since magno-cells saturate at approximately 10% contrast. In still further embodiments, the complexity level may additionally or alternatively increase by changing the pattern's speed of movement after approximately every four complexity levels. For example, the pattern's speed of movement may increase from approximately 5 Hz to approximately 5.7 Hz to approximately 6.7 Hz to approximately 8 Hz (in theta range) to approximately 10 Hz to approximately 13.3 Hz (in alpha range). This may allow the PATH module 208 to challenge the user as the training progresses. In some embodiments, faster speeds of movement, approximately 10 Hz to approximately 13.3 Hz, may be too fast to be trained on until slower speeds of movement are trained.

In some embodiments, the PATH module 208 may present the status of the training to the patient/user. For example, the PATH module 208 may present the number of patterns reviewed in the session, the remaining number of patterns, current score, personal best score, and the number of rewards or some combination thereof. In some embodiments, the user may also restart the threshold measurement by pushing the ‘x’ key to try and get a lower contrast threshold or push the space bar to see a new pattern when distracted or confused by the direction of movement. This may enable the patient to feel a sense of accomplishment as they progress through the training.

Once all complexity levels of the Motion program are completed, the user may progress onto the next program, the MotionMemory program. In some embodiments, the MotionMemory program may have a predetermined number of levels. This may be between approximately 16 and approximately 24 levels. The user can set the complexity level of both Motion and MotionMemory to be from 1 up to 24, enabling the same complexity level to be repeated. In some embodiments, when 24 complexity levels are finished, the user may return to a lower complexity level and restart the progression from low to high complexity levels for each training cycle. In some embodiments, a training cycle may include approximately twenty patterns, completed ten at a time in the current embodiment. In other embodiments, the MotionMemory may have approximately 16 complexity levels. In other embodiments, the MotionMemory may have approximately 24 complexity levels. In some embodiments, MotionMemory may require signaling the direction two separate patterns move, one after the other, by pushing one of four arrow keys: the left arrow key if the stripes moved left and then right, the right arrow key if the stripes moved right and then left, the up arrow key if the stripes only moved right, and the down arrow key if the stripes only moved left. In some embodiments, instead of pushing one of the four arrow keys, the left and right arrow keys can be pushed in sequence matching the direction of movement of the two patterns just seen, one after the other. Thus, in some instances, MotionMemory may engage working memory more than required for the Motion program. Each threshold in Motion and MotionMemory program may require between approximately twenty to forty trials to complete.

In some instances, a score may be provided to make the training more game-like. For example, the lower the contrast threshold, the higher the score is, see FIG. 15. Other motivational strategies in the training may include earning a fish for each low contrast threshold (approximately <=1% contrast), and at the end of each training cycle a graph displaying original, current, and optimal contrast sensitivity function for each test frequency, a star, or some combination thereof for each complexity level completed may be presented. This summary data may be stored in the patient module 202. In some embodiments, the user may access their rewards by clicking on Performance tab, see FIG. 9, on dashboard, see FIG. 16. In some instances, individual data may be accessed by clicking on a data tab, see FIG. 10, on dashboard, see FIG. 16. By clicking on each contrast threshold in the data tab, a graph may be presented showing performance on each trial. This may provide easy access to a subject's performance, see FIG. 11. In some embodiments, the data may be accessed by caregivers or health care providers.

In some instances, a dashboard, see FIG. 16, may display programs for user and training videos. A dashboard may enable both therapist/teacher and students to access data, as well as current complexity level for both Motion and MotionMemory programs, number of sessions used, and number of sessions still available. This may be how a user accesses PATH neurotraining online. Therefore, in some embodiments, the training videos may enable the programs to be completed without other assistance. By automating the task, the PATH neurotraining may be performed online. FIG. 16 is an exemplary dashboard. In some embodiments, more or less information may be presented. In still further embodiments, the exemplary dashboard may be presented in different order, with different backgrounds, or by accessing different screens.

In some embodiments, the PATH neurotraining videos may be presented to the user in a 12-bit grayscale. In some embodiments, 12-bit grayscale may allow for finer gradations between light and dark areas, potentially revealing more detail in images, enabling lower contrasts to be displayed, those below approximately 1%, needed to measure low contrast thresholds. In still further embodiments, the videos may be presented by using low gray scaled patterns. For example, the low gray scaled patterns presented to the user may be characterized by a limited number of distinct grayscale values, creating a sense of simplicity and texture. This may create the illusion of 12 bits of grayscale available by adjusting the color guns separately instead of adjusting the color guns together using only 8 bits of grayscale available when adjusting the color guns normally. This may enable the videos to play on a wider range of devices. In some embodiments, the videos may be coded using HTML5, JavaScript, or other programing languages able to achieve low gray scale patterns or 12-bit patterns, and the like on all types of computers and tablets.

Referring back to FIG. 2, the memory module 210 may sequentially follow the training provided by the Path module 208. The memory module 210 may present a sequence of digits to the user. The digits may comprise large black numbers on a white background. In some instances, this may be reversed, the digits may be large white numbers on a black background. In another embodiment, the digits and background may be any high contrast black and white or white on black combination. In another embodiment, the digits and background may be any high contrast color combination. In some embodiments, each digit may be presented for approximately 500 msec in the center of a screen. The memory module 210 may cycle through a predetermined number of digits before completing the cycle. The number of digits may increase from five digits up to ten digits, increasing by one digit each time, each sequence in different orders, with no repeating digits. Studies have shown ten digits is near the limit of remembering in an approximately 500 msec interval, since serial search (sequential processing) takes approximately 20-45 msec per item, and previous items being presented must be kept in working memory. However, in some embodiments, the memory module 210 may increase beyond ten digits. After the memory module 210 presents the sequence, a blank screen may be shown. The user may be prompted to recount the digits just seen and in the correct order. In some embodiments, this is prompted electronically. The memory module 210 may receive user input via either voice command, key strike, or another input. In some embodiments, the user may write the numbers down on a piece of paper. Once the user has finished entering the sequences of numbers, from 5 to 10 digits for example, the user may prompt the memory module 210 for an answer key. This may enable the subject to learn any mistakes. The memory module 210 may then move on to the next sequence of numbers repeating the same process. In some embodiments, subjects may recall the number of digits for up to four sequences of increasing digits for a total of approximately ten minutes. In other embodiments, the user types in the digits they just saw into an electronic notebook, and the computer corrects each sequence, providing the number of correct sequences out of six (increasing from five up to six digits) to the user and storing this result in the user's database.

Sequential processing uses the functional anatomy of the claustral connections of items being processed serially, such that cross-frequency coupling between low frequency signals from the claustrum and higher frequency oscillations in the cortical areas is an efficient means of the claustrum modulating neural activity across multiple brain regions in synchrony. Cross-frequency coupling is being recognized as an efficient means of communication between two cortical areas and it is likely to play a crucial role in mediating working memory and in enabling learning. Therefore, in some embodiments, the combination of the PATH module 208 and the memory module 210 may increase neural feedback, measured by coupled theta/gamma and alpha/gamma oscillations and visual working memory, memory recruitment, and memory storage. In some embodiments, including the memory module 210 strategy may cause the visually based intervention training to take longer than a predetermined time period.

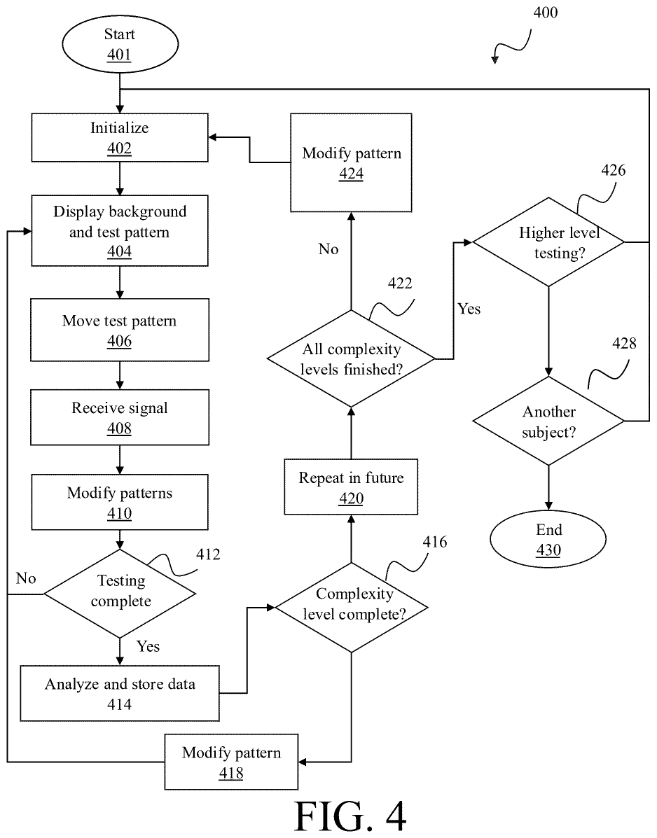

FIG. 4 is a flowchart illustrating steps in an exemplary methodology for measuring and improving contrast sensitivity and cognitive skill of a subject in accordance with the present disclosure. For clarity, the method 400 is described below with reference to aspects of one or more of the systems described herein.

At block 401, the method 400 may begin the testing pattern. At block 402, the method may initialize the testing patterns. The method 400 may begin all pattern components for the test and background patterns from a predetermined range of contrasts, spatial and temporal frequencies. In some embodiments, the method 400 may then generate image files for the test and background pattern in the form of pixel maps or pixmaps.

At block 404, the method 400 may display a background and test pattern. At block 406, the method 400 may move the test pattern either in one direction or the opposite direction. After viewing the test pattern movement, the subject may signal which direction the test pattern moved. At block 408, the method 400 may receive this signal. Then at block 410, the method may adjust the contrast of the test pattern which may determine the contrast threshold. At block 412, the method 400 may determine if the testing is completed. If testing is not completed, the method 400 may return to block 404. If the testing is completed, the method may move on to block 414.

In one embodiment, at blocks 402-412, the method 400 may measure contrast sensitivity for motion discrimination and may determine and, as shown in studies, may improve the subject's motion discrimination contrast sensitivity function (CSF) at both low and high levels in the dorsal stream. To measure the CSF, the method 400 may implement a staircase procedure to measure a contrast-sensitivity threshold for each spatial frequency of the test pattern at each spatial frequency of the background.

In some embodiments, the method 400 may measure contrast sensitivity and may include a plurality of preliminary initial steps, as just described. In some embodiments, the method 400 may measure and improve contrast sensitivity for motion discrimination, which specifically includes direction (i.e. left-right) discrimination. At block 414, the method 400 may store the data for the specified test-pattern spatial frequency to generate a CSF for the specified test-pattern spatial frequency, as exemplified in FIG. 11.

At block 416, the method 400 may determine if the testing for all of the predetermined background-test pattern spatial frequencies at each complexity level is complete. (If the subject has not been tested for all of the predetermined test-pattern spatial frequencies, after completing the testing for a particular test-pattern, at block 418, the method 400 may modify contrast, and/or spatial frequencies and/or temporal frequency of the test and background patterns. For example, the spatial frequency may be increased or decreased with the preferred predetermined range of approximately 0.25 cyc/deg, approximately 0.5 cyc/deg, approximately 1 cyc/deg, and approximately 2 cyc/deg, and the method may then return to block 404. This process may be repeated until the method 400, at block 416, has determined all complexity levels are completed. Then the method 400 may advance to block 420.

At block 420, the method 400 may have the test subject repeat the process (blocks 404 through block 416). At block 422, the method 400 may determine if all complexity levels are completed. If all complexity levels are not complete, the method 400 may advance to block 424 where the method 424 may modify the pattern before returning to block 402 and initializing the method 400 with the modified pattern.

In some embodiments, at the end of a staircase run for the particular pattern being used either at block 414 or block 424, the method 400 may adjust the background contrast, the multiplicity of background frequencies, the temporal frequency of test and background patterns, or some combination thereof in response to a correct signal. At block 424, the method 400 may increase the level of complexity by modifying the background contrast, and/or test and background spatial frequencies, and/or test pattern's temporal frequency, or some combination thereof.

Once the predetermined levels of complexity are completed, the method 400 may progress to block 426. At block 426, the method 400 may either 1) return to previous program at a lower level of complexity or 2) test higher levels of processing in the dorsal stream by increasing the task complexity. For example, when several directions of moving test pattern are displayed, the method 400, as described, may measure the contrast sensitivity of the test pattern for motion discrimination at higher levels in the dorsal stream. The contrast of the background pattern may equal initial contrast of the test pattern (5%), or be 2 (10%) or 4 (20%) times higher. When a sequence of discrete movements is displayed, the contrast of the test pattern may be increased if both pattern presentations are not identified correctly. In one embodiment, once a subject is in the two-alternative staircase, the contrast of the test pattern may be decreased only when the directions of motion of all patterns in the sequence of discrete movements are identified correctly three times in a row. In this embodiment, separate keys may be used for each of the pattern alternatives. For example, when two patterns are presented consecutively, then the left arrow key is pushed when the patterns moved left and then right, the right arrow key is pushed when the patterns moved right and then left, the up-arrow key when both patterns moved right, and the down arrow key, when both patterns moved left.

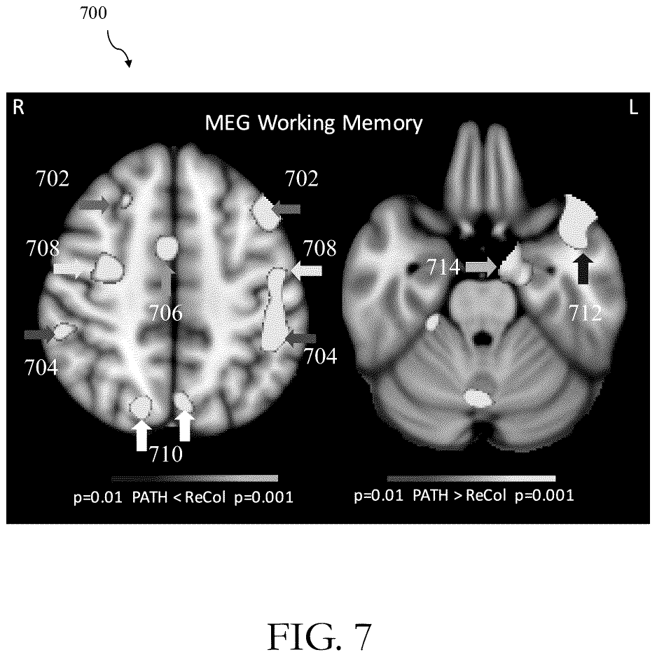

Although the method 400 has been described in relation to the contrast sensitivity for discriminating movement, for example, and to improve the timing between the dorsal and ventral streams at different levels of cortical processing to improve speed of processing, and widen the attention gateway, it also improves the executive control network. FIG. 7 is an image of MEG working memory 700. As shown in FIG. 7, the method 400 improves the dorsal lateral prefrontal cortex 702 and the posterior parietal cortex 704—the two hubs of the executive control network, the anterior cingulate cortex 706—a hub of the salience network controlling attention, primary motor cortex 708, the superior occipital gyri 710, left anterior temporal lobe 712, and left hippocampus 714 and cerebellum 716.

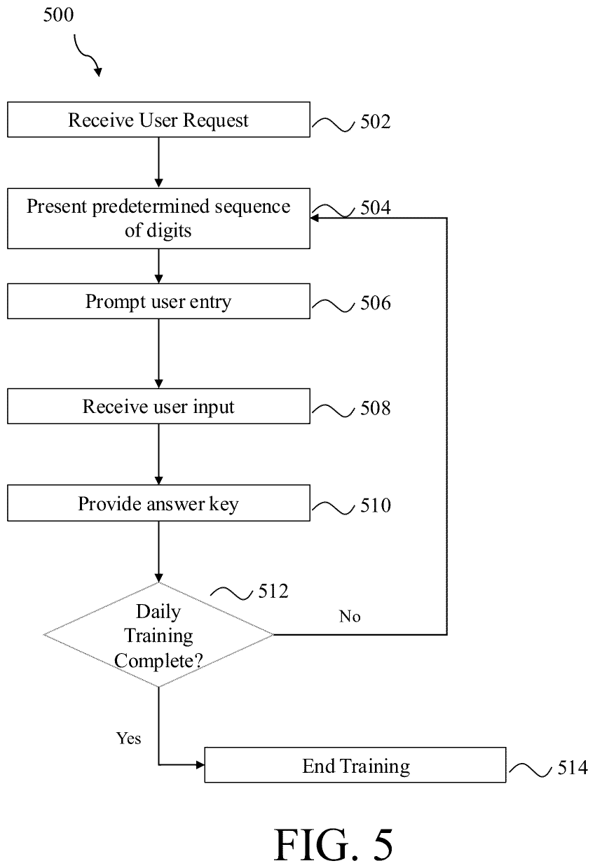

FIG. 5 is a flowchart illustrating steps in an exemplary methodology for measuring and improving working memory. For clarity, the method 500 is described below with reference to aspects of one or more of the systems described herein.

At block 502, the method 500 may include receiving a user request. The user request may be a request to begin the memory training. This may prompt the method 500, at block 504, to present a predetermined sequence of digits to the user. The digits may be presented as large black numbers on a white background or large white numbers on a black background. The numbers may be present from approximately 500 msec before advancing to the next number in the sequence. The number of digits may start at approximately five digits and increase a single digit each time the sequence begins to a maximum of ten digits. In this embodiment, a user may begin with a five-digit sequence and repeat the sequence with an increasing number of digits presented. The user may perform this task for either a predetermined duration of time or a predetermined quantity of sequences. In some embodiments, the method 500 may stop the user at whichever stopping point arrives first, either a time limit or a quantity of sequences.

After each sequence, at block 506, the method 500 may prompt the user to recount the number sequence in order. This may be done electronically or manually depending on the user's dexterity and preferences. At block 508, once the method 500 has received the user input, and the user has finished the sequence of from 5 to 10 digits, the method may then, at block 510, provide the answer key for the most recent sequence of from 5 to 10 digits presented to the user. The user may then correct their mistakes manually or the method 500 may electronically provide corrections to the user.

At block 512, the method 500 will determine if the user has completed their daily training. As mentioned previously, the user may be required to perform the training for a predetermined period of time each day and/or perform a predetermined quantity of sequences each training cycle. If the user has completed their training, then at block 514, the method may end the training. If the user has not completed their training, the method 500 may return to block 504 and continue to present the next sequence of numbers to the user. The method 500 will continue to repeat this process until the training is complete.

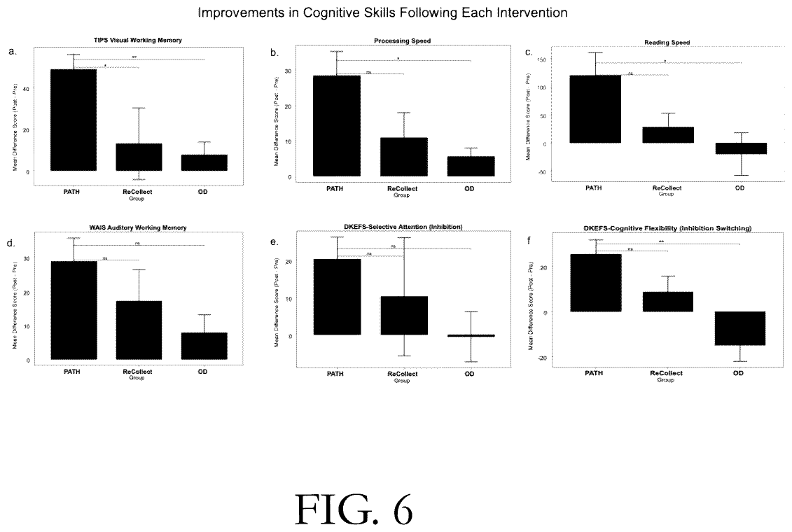

FIG. 6 illustrates six graphs of the cognitive skills that improved, plotting standardized post-test scores minus pre-test scores, error bars corresponding to the standard error (SE) scores, for each of the three interventions being investigated: PATH for twenty minutes followed by ten minutes of digit memory exercises, Orientation Discrimination (OD) for twenty minutes followed by ten minutes of digit memory exercises, and ReCollect, a conventional N-Back task, for thirty minutes. These interventions were completed for thirty-six sessions three times a week (twelve weeks duration). Note: *=p<0.05; **=p<0.01; error bars indicate ±1 SEM. For all six cognitive skills, The PATH group exhibited the largest improvements in standardized percentile scores, followed by the ReCollect group. Minimal improvement was observed in the OD group. Individual variability in response to training is shown by SE bars.

FIG. 6A graphs post-minus pre-test visual working memory (VWM) scores that represent performance on the Test of Information Processing Skills (TIPS) for each of the three interventions. On average, the PATH group exhibited a 49% improvement in VWM, compared to 13% for ReCollect and 8% for OD groups. To examine the impact of group assignment on changes in visual working memory performance, a 2 (Time: Pre, Post)×3 (Group: PATH, ReCollect, OD) mixed-model ANOVA was conducted. Results from the mixed-model ANOVA indicated significant main effects of Group, F(2, 21)=4.38, p=0.026, η²p=0.29, and Time, F(1, 21)=14.67, p<0.001, η²p=0.41, as well as a significant Group×Time interaction, F(2, 21)=5.67, p=0.011, η²p=0.35. To further clarify the nature of this interaction, a one-way ANOVA was performed on the change scores (Post-Pre), which revealed a significant effect of Group, F(2, 21)=5.67, p=0.011, η²p=0.35. In summary, the hypothesis that group assignment would predict post-training gains in visual working memory was supported, with the PATH group exhibiting the most pronounced improvement following intervention training. Moreover, the PATH group showed a statistically significant advantage over both the ReCollect and the OD group.

FIG. 6B graphs post-minus pre-test processing speed scores that represent performance on the Wechsler Adult Intelligence Scale-IV (WAIS-4) Digit Coding and Symbol Search subtests that provides a standardized processing speed index (PSI) score. On average, the PATH group exhibited a 28% improvement in PSI, compared to 11% for ReCollect and 6% for OD groups. To examine the impact of group assignment on changes in processing speed, a 2 (Time: Pre, Post)×3 (Group: PATH, ReCollect, OD) mixed-model ANOVA was conducted. Results from the mixed-model ANOVA indicated significant main effects of Group, F(2, 21)=15.41, p<0.001, η²p=0.59, and Time, F(1, 21)=12.66, p=0.002, η²p=0.38. The Group×Time interaction approached significance, F(2, 21)=3.33, p=0.056, η²p=0.24. To further clarify the nature of this effect, a one-way ANOVA was performed on the change scores (Post-Pre), which also approached significance for Group, F(2, 21)=3.33, p=0.056, η²p=0.24. In summary, the hypothesis that group assignment would predict post-training gains in processing speed was supported with the PATH group exhibiting the most pronounced improvement following intervention training, the PATH group showing a statistically significant advantage over the OD group.

FIG. 6C graphs post-minus pre-test reading speed scores (words per minute) that represent performance on a computer-based reading test that presented six words at a time from the book Cheaper by the Dozen at increasingly faster speeds until six words could only be read correctly 79% of the time. On average, the PATH group exhibited a 120 words/min improvement in Reading Speed, compared to 29 words/min for ReCollect and −20 words/min for OD groups. To examine the impact of group assignment on changes in computer-based reading speed (words per minute; WPM), a 2 (Time: Pre, Post)×3 (Group: PATH, ReCollect, OD) mixed-model ANOVA was conducted. Results from the mixed-model ANOVA indicated a significant main effect of Group, F(2, 21)=3.89, p=0.037, η²p=0.27. The main effect of Time did not reach statistical significance, F(1, 21)=2.97, p=0.099, η²p=0.12. The Group×Time interaction approached significance, F(2, 21)=3.28, p=0.058, η²p=0.24. To further clarify the group effect, a one-way ANOVA was performed on the change scores (Post-Pre), which revealed that the effect of Group on change score also approached significance, F(2, 21)=3.28, p=0.058, η²p=0.24. In summary, the hypothesis that group assignment would predict post-training gains in reading speed was supported with the PATH group exhibiting the most pronounced improvement following intervention training, the PATH group showing a statistically significant advantage over the OD group.

FIG. 6D graphs post-minus pre-test auditory working memory (AWM) scores, that represent performance on the WAIS-4 Digit Span and Letter-Number sequencing subtests that provides a standardized auditory working memory index score. On average, the PATH group exhibited a 29% improvement in AWM, compared to 17% for ReCollect and 8% for OD groups. In the analysis of WAIS AWM, only an effect of Time emerged in the ANOVA, f(1,21)=15.08, p<0.001. There was no main effect of Group, or interaction with Time and Group (p>0.05). The hypothesis that group assignment would predict post-training gains in auditory working memory was supported with the PATH group exhibiting the most pronounced improvement following intervention training. In the analysis of change scores, only PATH versus OD emerged as marginally significant (p=0.064), suggesting that PATH only improves significantly more than OD.

FIG. 6E graphs post-minus pre-test selective attention scores that represent performance on the Delis-Kaplan Executive Function System (DKEFS) Color-Word Interference Inhibition subtest. On average, the PATH group exhibited a 20% improvement in Selective Attention, compared to 10% for ReCollect and −0.5% for OD groups. The hypothesis that group assignment would predict post-training gains in selective attention was supported with the PATH group exhibiting the most pronounced improvement following intervention training.

FIG. 6F graphs post-minus pre-test cognitive flexibility scores that represent performance in the DKEFS Color-Word Interference Inhibition-Switching subtest. On average, the PATH group exhibited a 26% improvement in Cognitive Flexibility, compared to 9% for ReCollect and −15% for OD groups. To examine the impact of group assignment on changes in DKEFS-Inhibition-Switching performance, a 2 (Time: Pre, Post)×3 (Group: PATH, ReCollect, OD) mixed-model ANOVA was conducted. Results from the mixed-model ANOVA indicated a significant main effect of Group, F(2, 21)=4.45, p=0.025, η²p=0.30, and a significant Group×Time interaction, F(2, 21)=8.28, p=0.002, η²p=0.44, but the main effect of Time was not significant, F(1, 21)=2.20, p=0.153, η²p=0.09. To further clarify the nature of this interaction, a one-way ANOVA was performed on the change scores (Post-Pre), which revealed a significant effect of Group, F(2, 21)=8.28, p=0.002, η²p=0.44. In summary, the hypothesis that group assignment would predict post-training gains in DKEFS-Inhibition Switching performance was supported, with the PATH group exhibiting the most pronounced improvement following intervention training, the PATH group showing a statistically significant advantage over the OD group.

Therefore, FIG. 6 shows that only when digit memory exercises follow PATH training, are cognitive skills improved significantly, i.e. when digit memory exercises follow OD training, these improvements were not measured. Higher significance levels were not measured, since only 24 mTBI participants were in this study. However, in spite of this small sample size, this study provides compelling evidence that PATH neurotraining followed by digit memory exercises, being delivered as a targeted mTBI intervention, yields substantial cognitive functioning improvements—most notably in visual and auditory working memory, processing speed, reading speed, selective attention, and cognitive flexibility. These behavioral gains are supported by converging neurophysiological evidence from MEG, demonstrating enhanced task-evoked activation and normalization of resting-state gamma oscillatory activity within distributed networks encompassing prefrontal, cingulate, parietal, occipital, temporal, motor, and cerebellar regions.

FIG. 7 illustrates neuroimaging results 700 from evoked MEG Working Memory (WM) N-back task, showing stronger (POST- vs PRE-treatment) responses in individuals with mTBI who finished the PATH treatment than those who went through the ReCollect treatment. Stronger responses were from: bilateral dorsal lateral PreFrontal Cortex: dlPFC 702; Anterior Cingulate Cortex: ACC 706, Posterior Parietal Cortex: PPC 704, primary motor cortex 708, superior occipital gyri 710, left anterior temporal lobe 712, left hippocampus 714, and cerebellum 716. PATH neurotraining improves cortical areas (anterior temporal lobe, left hippocampus, primary motor cortex, and cerebellum) not found except when digit memory task follows PATH neurotraining.

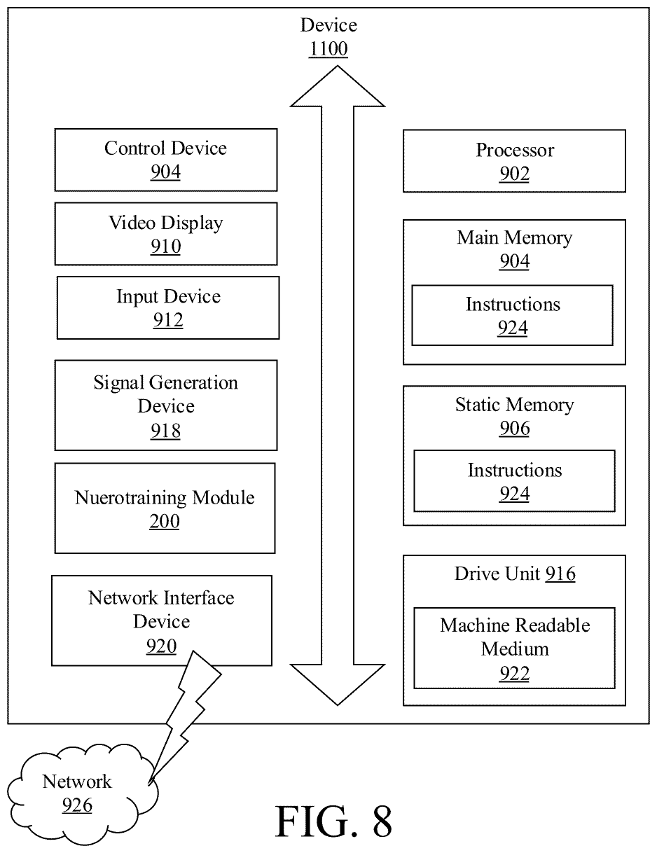

FIG. 8 is a diagram displaying various components of an example device 1100. The device 1100 may include a set of instructions causing the device 1100 to perform any one of more of the methodologies described herein. In some embodiments, the device 1100 may be an example of devices 102, 122 as shown in FIG. 1. In alternative embodiments, the device 1100 may operate as a standalone device or may be connected (e.g., networked) to other machines. In a networked deployment, the device 1100 may operate in the capacity of a server or a client machine in server-client network environment, or as a peer machine in a peer-to-peer (or distributed) network environment. The device 1100 may be a server computer, a client computer, a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single device 1100 is illustrated, the term “machine” shall also be taken to include any collection of machines that individually or jointly execute a set (or multiple sets) of instructions to perform any one or more of the methodologies discussed herein.

The device 1100 includes a processor 902 (e.g., a central processing unit (CPU) a graphics processing unit (GPU) or both), a main memory 904 and a static memory 906, which communicate with each other via a bus 908. The device 1100 may further include a video display unit 910 (e.g., a liquid crystal display (LCD) or a cathode ray tube (CRT)). The device 1100 also includes an alphanumeric input device 912 (e.g., a keyboard), a cursor control device 914 (e.g., a mouse), a disk drive unit 918, a signal generation device 918 (e.g., a speaker) and a network interface device 920.

The disk drive unit 916 includes a machine-readable medium 922 on which is stored one or more sets of instructions (e.g., software 924) embodying any one or more of the methodologies or functions described herein. The software 924 may also reside, completely or at least partially, within the main memory 904 and/or within the processor 902 during execution thereof by the device 1100, the main memory 904 and the processor 902 also constituting machine-readable media.

The software 924 may further be transmitted or received over a network 926 via the network interface device 920.

While the machine-readable medium 922 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. The term “machine-readable medium” shall accordingly be taken to include, but not be limited to, solid-state memories, optical and magnetic media, and carrier wave signals.

A person skilled in the art will be able to practice the present invention after careful review of this description, which is to be taken as a whole. Details have been included to provide a thorough understanding. In other instances, well-known aspects have not been described, in order to not obscure unnecessarily this description.

Some technologies or techniques described in this document may be known. Even then, however, it is not known to apply such technologies or techniques as described in this document, or for the purposes described in this document.

This description includes one or more examples, but this fact does not limit how the invention may be practiced. Indeed, examples, instances, versions or embodiments of the invention may be practiced according to what is described, or yet differently, and also in conjunction with other present or future technologies. Other such embodiments include combinations and sub-combinations of features described herein, including for example, embodiments that are equivalent to the following: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In general, the present disclosure reflects preferred embodiments of the invention. The attentive reader will note, however, that some aspects of the disclosed embodiments extend beyond the scope of the claims. To the respect that the disclosed embodiments indeed extend beyond the scope of the claims, the disclosed embodiments are to be considered supplementary background information and do not constitute definitions of the claimed invention.

In this document, the phrases “constructed to”, “adapted to” and/or “configured to” denote one or more actual states of construction, adaptation and/or configuration that is fundamentally tied to physical characteristics of the element or feature preceding these phrases and, as such, reach well beyond merely describing an intended use. Any such elements or features can be implemented in a number of ways, as will be apparent to a person skilled in the art after reviewing the present disclosure, beyond any examples shown in this document.

Incorporation by reference: References and citations to other documents, such as patents, patent applications, patent publications, journals, books, papers, web contents, have been made throughout this disclosure. All such documents are hereby incorporated herein by reference in their entirety for all purposes.

Parent patent applications: Any and all parent, grandparent, great-grandparent, etc. patent applications, whether mentioned in this document or in an Application Data Sheet (“ADS”) of this patent application, are hereby incorporated by reference herein as originally disclosed, including any priority claims made in those applications and any material incorporated by reference, to the extent such subject matter is not inconsistent herewith.

Reference numerals: In this description a single reference numeral may be used consistently to denote a single item, aspect, component, or process. Moreover, a further effort may have been made in the preparation of this description to use similar though not identical reference numerals to denote other versions or embodiments of an item, aspect, component or process that are identical or at least similar or related. Where made, such a further effort was not required, but was nevertheless made gratuitously so as to accelerate comprehension by the reader. Even where made in this document, such a further effort might not have been made completely consistently for all of the versions or embodiments that are made possible by this description. Accordingly, the description controls in defining an item, aspect, component or process, rather than its reference numeral. Any similarity in reference numerals may be used to infer a similarity in the text, but not to confuse aspects where the text or other context indicates otherwise.

The claims of this document define certain combinations and subcombinations of elements, features and acts or operations, which are regarded as novel and non-obvious. The claims also include elements, features and acts or operations that are equivalent to what is explicitly mentioned. Additional claims for other such combinations and subcombinations may be presented in this or a related document. These claims are intended to encompass within their scope all changes and modifications that are within the true spirit and scope of the subject matter described herein. The terms used herein, including in the claims, are generally intended as “open” terms. For example, the term “including” should be interpreted as “including but not limited to,” the term “having” should be interpreted as “having at least,” etc. If a specific number is ascribed to a claim recitation, this number is a minimum but not a maximum unless stated otherwise. For example, where a claim recites “a” component or “an” item, it means that the claim can have one or more of this component or this item.

In construing the claims of this document, the inventor(s) invoke 35 U.S.C. § 112(f) only when the words “means for” or “steps for” are expressly used in the claims. Accordingly, if these words are not used in a claim, then that claim is not intended to be construed by the inventor(s) in accordance with 35 U.S.C. § 112(f).

What is claimed is:

1. A method to improve cognitive skills of a user, the method including:
displaying, on a monitor, a background and a test pattern, wherein the background includes a pattern which has visibly lighter portions and darker portions that visibly vary in luminance across a spatial dimension at multiple spatial frequencies, and wherein the test pattern includes a contrast between a lighter portion and a darker portion moving in one or more directions within the boundaries of the background;
receiving a communication from the user through a user interface indicating the one or more directions in which the test pattern appears to the user to have moved;
providing feedback to the user when an incorrect communication is received;
repeating the displaying and the receiving steps associated with the background and the test pattern a predetermined number of pattern sequences;
determining when the predetermined number of pattern sequences is complete;
presenting, on the monitor, a predetermined sequence of digits to the user when the predetermined number of pattern sequences is complete;
obtaining a communication from the user through the user interface wherein the user enters the predetermined sequence of digits in the order presented; and
correcting the entries based at least in part on the correct order of sequence of digits displayed.

2. The method of claim 1, wherein each digit separated by at least 10 msec.

3. The method of claim 2, further comprising:
presenting, on the monitor, a further set of predetermined sequence of digits to the user after a prior set of predetermined sequences of digits is complete, where the further set of predetermined number of digits increases by one digit each time, the further set of predetermined sequences of digits ending when a total of 10 digits is presented.

4. The method of claim 3, where each further set of predetermined sequence of digits presents no repeating digits and no repeating sequences.

5. The method of claim 4, wherein a quantity of the further set of predetermined sequence of digits is greater than a quantity of the prior set of predetermined sequence of digits.

6. The method of claim 1, further including:
presenting a second predetermined sequence of digits to the user after the first entries are corrected.

7. The method of claim 1, wherein the test pattern is displayed in low contrast grayscale below 10% contrast, and the background pattern contrast is one of equal to 20% contrast and below 20% contrast.

8. The method of claim 1, further comprising:
receiving a request from the user to repeat a process, wherein the process is one of the pattern sequence and digit sequence.

9. The method of claim 8, wherein one of the pattern sequence and digit sequence process repeats at a different complexity level.

10. The method of claim 1, further comprising:
recording user entries in the associated user profile on the encrypted server, wherein the entries are stored in a structured hierarchical format.

11. The method of claim 10, further comprising:
compiling the user personal information and user entries into a dashboard.

12. The method of claim 1, further comprising:
enabling a second user to remotely access the pattern and digit sequence presenting and user response.

13. The method of claim 1, further comprising:
calibrating the monitor to test the contrast of the lighter portion and the darker portion of the test pattern.

14. The method of claim 1, further comprising:
displaying, on the monitor, the contrast pattern in 12-bit color grayscale.

15. The method of claim 14, further comprising:
converting 8-bit color grayscale monitor to display the contrast pattern in 12-bit color grayscale.

16. A method to remediate cognitive skills, the method including:
presenting, on a monitor, a predetermined sequence of digits to a user;
obtaining a communication from the user through a user interface wherein the user enters the predetermined sequence of digits in the order presented; and
correcting the entries based at least in part on the correct order of sequence of digits displayed.

17. The method of claim 16, wherein the predetermined number of digits is five digits, each digit separated by at least 10 msec, increasing by one digit each time up to a total of 10 digits, presenting no repeating digits and no repeating sequences.

18. The method of claim 16, further comprising:
determining when the user has completed a predetermined number of sequence recounting cycles; and
ending the training when the predetermined number of sequence recounting cycles is completed.

19. The method of claim 16, further comprising:
displaying a second set of predetermined sequence of numbers to the user when the user has not completed the predetermined number of sequence recounting cycles.

20. A method to improve cognitive skills of a user, the method including:
displaying, on a monitor, a background and a test pattern, wherein the background includes a pattern which has visibly lighter portions and darker portions that visibly vary in luminance across a spatial dimension at multiple spatial frequencies, and wherein the test pattern includes a 12 bit grayscale contrast between a lighter portion and a darker portion moving in one or more directions within the boundaries of the background;
receiving a communication from the user through a user interface indicating the one or more directions in which the test pattern appears to the user to have moved;
providing feedback to the user when an incorrect communication is received;
repeating the displaying and the receiving steps associated with the background and the test pattern a predetermined number of pattern sequences;
determining when the predetermined number of pattern sequences is complete;
presenting, on the monitor, a predetermined sequence of digits to the user one digit at a time, displaying each digit for 500 ms, wherein the predetermined sequence of digits is displayed in black lettering on a white background, when the predetermined number of pattern sequences is complete;
obtaining a communication from the user through the user interface wherein the user enters the predetermined sequence of digits in the order presented; and
correcting the entries based at least in part on the correct order of sequence of digits displayed.
